(19) United States
(12) Patent Application Publication
(10) Pub. No.: US 2008/0267344 A1
(43) Pub. Date: Oct. 30, 2008

(54) Signal Processing Unit for Producing Images

(75) Inventors: Thomas Koehler, Norderstedt (DE); Michael Grass, Buchholz in der Nordheide (DE)

Correspondence Address: PHILIPS INTELLECTUAL PROPERTY & STANDARDS, 595 MINER ROAD, CLEVELAND, OH, 44143, US

(73) Assignee: Koninklijke Philips Electronics N.V., Eindhoven (NL)

(21) Appl. No.: 12/093,181

(22) PCT Filed: Nov. 6, 2006
(86) PCT No.: PCT/IB06/54119

§ 371 Date: May 9, 2008

(30) Foreign Application Priority Data
Nov. 10, 2005 (EP) 05110586.4

(51) Int. Cl. A61B 6/03

(52) U.S. Cl. 378/8

(57) ABSTRACT

An exemplary embodiment of the invention provides a signal processing unit for producing images of an object under examination based on data signals of a tomography system, the signal processing unit comprising a processor and an input interface, wherein the input interface is adapted to receive measured data signals. Furthermore, the processor is adapted to generate a pulmonary gating signal based on said measured data signals and is further adapted to generate an image based on said measured data signals by using the pulmonary gating signal.

The invention relates to a signal processing unit for producing images, a tomography system comprising a signal processing unit, a method for producing an image, a computer readable medium and a program element.

Signal processing units are used in a plurality of fields. One field is Computer Tomography (CT) and in particular so-called retrospective pulmonary gated Computer Tomography. Retrospective pulmonary gated Computer Tomography is an acquisition and analyzing mode of increasing interest. In such retrospective pulmonary gated CT devices a signal processing of taken data from an object will be performed under consideration of the motion of the object during the imaging by taking into consideration a gating signal. For example, while generating an image of a chest or abdomen of a subject only data referring to a determined point in a breathing cycle of the subject is used for generating the image. This is done since breathing will shift the outer position of the chest and or inner organs. In such a way it is possible to reduce artefacts caused by movement. One particular concern in pulmonary gating of CTs is the gating device itself.

A conventional gating device is disclosed in US 2004/0081269, for example. The CT system disclosed in that document comprises a gantry having an x-ray source, a radiation detector array, a patient support structure and a patient cavity. For realizing a pulmonary (respiratory) gating of the x-ray source the system comprises a special sensor system responsive to the patient's respiratory cycle and generating a zero phase pulse. This pulse gates the generation of the radiation beam and of the scanning data acquisition, and is also used as a gate signal for the signal processing and image generation in a signal processing unit.

However, there may be a need for another signal processing unit for producing, for another tomography system comprising a signal processing unit and for another method for producing an image.

This need may be met by a signal processing unit, a tomography system comprising a signal processing unit, a method for producing an image based on data signals of a tomography system, a computer readable medium, and a program element according to the independent claims.

An exemplary embodiment of the invention provides a signal processing unit for producing images of an object under examination based on data signals of a tomography system, the signal processing unit comprises a processor and an input interface, wherein the input interface is adapted to receive measured data signals. Furthermore, the processor is adapted to generate a pulmonary gating signal based on said measured data signals and is further adapted to generate an image based on said measured data signals by using the pulmonary gating signal.

Moreover, an exemplary embodiment relates to a tomography system, the system comprises a signal processing unit and a tomography device having a radiation source and a radiation detector, wherein the radiation detector is adapted to measure data signals based on radiation emitted by the radiation source after having passed an object under examination. Furthermore, the signal processing unit is adapted to generate a pulmonary gating signal based on the measured data signals and the signal processing unit is further adapted to generate images based on the measured data signals while utilizing the pulmonary gating signal. The radiation detector can be formed of a single radiation sensor, a plurality of radiation sensors or a sensor array.

Further, an exemplary embodiment relates to a method for producing an image based on data signals of a tomography system the system comprises a tomography device having a radiation source and a radiation detector. The method comprising measuring data signals, which data signals are based on radiation emitted by the radiation source after having passed an object under examination, by using the radiation detector. The method further comprises generating a pulmonary gating signal based on said measured data signals and generating an image based on said measured data signals by using the pulmonary gating signal.

Furthermore, an exemplary embodiment relates to a computer readable medium in which a program for producing an image based on data signals of a tomography system having a radiation source and a radiation detector is stored. The program, when executed by a processor, is adapted to control a method comprising measuring data signals, which data signals are based on radiation emitted by the radiation source after having passed an object under examination, by using the radiation detector. Further, the method comprises generating a pulmonary gating signal based on said measured data signals and generating an image based on said measured data signals by using the pulmonary gating signal.

An exemplary embodiment relates to a program element for producing an image based on data signals of a tomography system having a radiation detector, which program, when executed by a processor, is adapted to control a method which comprises measuring data signals, which data signals are based on radiation emitted by the radiation source after having passed an object under examination, by using the radiation detector. Further, the method comprises generating a pulmonary gating signal based on said measured data signals, and generating an image based on said measured data signals by using the pulmonary gating signal.

A characteristic feature according to the present invention may be that intrinsic information from measured data signals, also-called projection data, are used to generate one or more pulmonary gating signals, in the following also called gating signal. Thus, it may be possible that no extra detector is needed to provide signals which can be used for generating a gating signal as it is necessary according to the prior art. Furthermore, the projection data may be used to calibrate a gating device, which may be external to the processing unit but may be a part of the tomography system or may not be a part of the tomography system. According to this application a gating signal may be a signal which is used in image generation to determine which measured data are used for the generation of the image and which measured data are not used for the generation of the image. The tomography system may be any type of tomograph, like a computer tomograph or a magnetic resonance tomograph.

The characteristic features according to the invention may have particularly the advantage that all information is derived from projection data, therefore the method and the signal processing unit may be very fast. The method and the signal processing unit may only use information which is intrinsic to the projection data. Thus, an acquisition protocol may become easier.

By using the projection data, i.e. the data which are also used for the generation of an image of the object under examination, for generating a gating signal a high redundancy of the projection data may be exploited. The generating and using of pulmonary gating in the generation of an image may be of particular interest when an image of a tumor situated in the thorax of a human is generated. The movement of the thorax may be indicative for the movement of the tumor situated in the thorax. Thus, it may be advantageous to use such a pulmonary gating signal than to use gating signals related to a heart beat. This may in particular relevant since the movement of the thorax, i.e. the movement of the chest wall, is a deliberately movement while the movement of the heart is a movement not subject to the free will. Thus, it may be possible that the movement of the tumor under examination, which is correlated to the movement of the chest, does not correlate to the heart beat.

Referring to the dependent claims, further preferred embodiments of the invention will be described in the following.

Next, preferred exemplary embodiments of the signal processing unit of the invention will be described. These embodiments may also be applied for the tomography system, the method, the computer readable medium, and the program element.

In another exemplary embodiment of the signal processing unit the processor is adapted to generate a gating signal which gating signal relates to minima of a periodic motion of the object.

In yet another exemplary embodiment the processor may be adapted to generate a gating signal which gating signal relates to maxima of a periodic motion of the object.

The above two embodiments may be combined in such a way that several gating signals or several groups of gating signals are generated by the processor. One of these signals or group of signals relates to a maximum of a periodic motion and one to a minimum of the periodic motion of the object under examination. Such an object may also be, beside other objects, a subject like a person from which a tomography is taken. In such case the periodic motion may be the movement of the chest, the abdomen and/or of inner organs of the person caused by breathing of the person. By using a gating signal which relates to the moving or the motion state of the object under examination artefacts in the image which artefacts are caused by the movement of the object, like moving caused by breathing, may be greatly reduced. Such breathing motion may create periodic motion or periodic variation of the upper chest wall and of the skin around the abdomen. By detection of the local minima and maxima of, e.g. the upper, chest wall the time for maximum inhalation and/or exhalation may be determined locally. Thus, a motion state may be estimated locally, while according to the state of the art, the breathing motion is monitored based on only one external sensor. Thus, the motion state determining using a signal processing unit according to the present invention may be better adapted to the true motion compared to signal processing units of tomography systems known in the prior art.

According to still another exemplary embodiment of the signal processing unit the processor is adapted to generate the pulmonary gating signal free of a determination of a centre of gravity of parallel projection.

By not using a determination of a centre of gravity of different parallel projections, which can be generated from the measured data, the generation may be simplified, e.g. one calculation can be omitted while generating the pulmonary gating signal.

Next, preferred exemplary embodiments of the tomography system of the invention will be described. These embodiments may also be applied for the signal processing unit, the method, the computer readable medium, and the program element.

In another exemplary embodiment of the tomography system the radiation source is rotatable. In a further exemplary embodiment the radiation source is adapted to rotate at a speed which is adapted to cover a periodic motion of the object. That is, the speed of rotation is great compared to the speed of the movement. In an exemplary embodiment the speed is higher than 1 rotation per second. That is, the radiation source will rotate at a speed of one, two, three or a higher number of rotations per second. By rotating at such a speed a sampling of data may be possible which is sufficient to cover breathing action in case the examined object is a subject in particular a person, which breathing usually is on a far courser time scale than one per second. According to this application the rotation speed is defined not only as the speed the radiation source rotates around its own axis but also the speed the radiation source rotates around the object under examination.

According to still another exemplary embodiment of the tomography system the tomography system is adapted to measure the data signals every 180° of the rotation of the radiation source.

With such a sampling every 180° of rotation of the radiation source it may be possible to double the sampling rate without increasing the rotational speed. For example in case the rotation speed is chosen to be short, like 0.5 seconds, measured data may be recorded which corresponds to a row of virtual parallel projection every 250 ms. Such a sampling may be sufficient to cover breathing motions. Furthermore, the sampling may be done with large spatial overlap. From each sampling a so called central row may be generated which may be almost a one-dimensional parallel projection of the object under examination. The central row after half a turn may contain almost the same parallel projection, shifted only by half a pitch. Taking the average of the two central rows, generated from the sampling at 0° and 180°, the full scan may give almost a two-dimensional parallel projection of the object under examination.

According to yet another exemplary embodiment the radiation is adapted in such a way that it emits a cone beam. Preferably, the radiation detector is adapted in such a way that the measured data signals are representing cone beam data.

Next, preferred exemplary embodiments of the method of the invention will be described. These embodiments may also be applied for the signal processing unit, the tomography system, the computer readable medium, and the program element.

According to another exemplary embodiment of the method the measured data signals are cone beam data and the method further comprises re-binning the cone-beam data into wedge geometry before generating the image based on the measured data signals.

When the image is generated by using the so-called wedge geometry it may be possible to takes the angle of a cone-beam into account and to enable handling of redundant data.

According to yet another exemplary embodiment the method further comprises the forming of a virtual parallel projection of the object under examination, based on the wedge geometry.

That is, as an intermediate step, one or more virtual parallel projections, which are similar to a so-called scanogram, may be computed from helical data, i.e. measured data which are taken along a helical path through the object under examination. This computation may be done by firstly re-binning the cone-beam data, i.e. the helical data, into wedge geometry. Then virtual parallel projections, e.g. projections along a central detector line, may be taken at positions which each relates to a further rotation of 180° of the radiation source. Thereby every other virtual parallel projection is mirrored and corrected for a shift relating to the movement of the object under examination in reference to the radiation detector, the so-called detector shift.

According to a further exemplary embodiment the gating signal is generated based on a plurality of virtual parallel projections.

By using more than one virtual parallel projections the temporal accuracy may be improved, e.g. using virtual parallel projections representing a projection angle of 0°, 45°, 90°, 135° and possibly other angles between 0° and 180°. By generating such additional parallel projections another signal curve may be achieved having the same features. When using a first parallel projection at 90° and second parallel projection at 0° two signal curves may be achieved wherein the samples of the second curve may be exactly between the samples of the first curve. Consequently it may be possible to get a doubling of motion signals if the two data sets are interleaved. Such an improvement might be also possible by using interpolation. The interpolation may be done by using measured data having a large spatial overlap which might be sufficient to interpolate a motion state, e.g. a breathing state.

According to yet still another exemplary embodiment of the method the gating signal is adapted to represent a motion state of the object under examination. In particular the motion state can be a maximum or a minimum of a periodic motion of the object under examination.

The present invention may be of particular interest in the field medical uses, e.g. in the field of so-called retrospective pulmonary gating of computer tomography. Preferably the present invention may be used in the field of cone-beam CT-systems with 16 or more detector rows, which CT-systems may make very low pitch helical acquisition feasible within reasonable time. A typical pitch may be about 0.1, e.g. 2.4 mm per rotation. If such a low pitch is combined with a short rotation time and a large spatial overlap an accurate motion state estimation may be possible using an interpolation of successive virtual parallel projections.

One aspect of the present invention might relate to a device-less pulmonary gating for computer tomography. That is, according to this aspect no extra sensors or detectors are used to generate a gating signal for the generation of the images of a subject under examination but the radiation detectors which are already used for taking the image data are used to provide the gating signal, i.e. the same data may be used to generate the gating signal and to generate the image. Thus, the high redundancy of the projection data in combination with a short rotation time compared with a typical breathing period of a subject under examination may be exploited to generate a gating signal.

The aspects defined above and further aspects of the invention are apparent from the examples of embodiment to be described hereinafter and are explained with reference to the examples of embodiment.

The invention will be described in more detail hereinafter with reference to examples of embodiment but to which the invention is not limited.

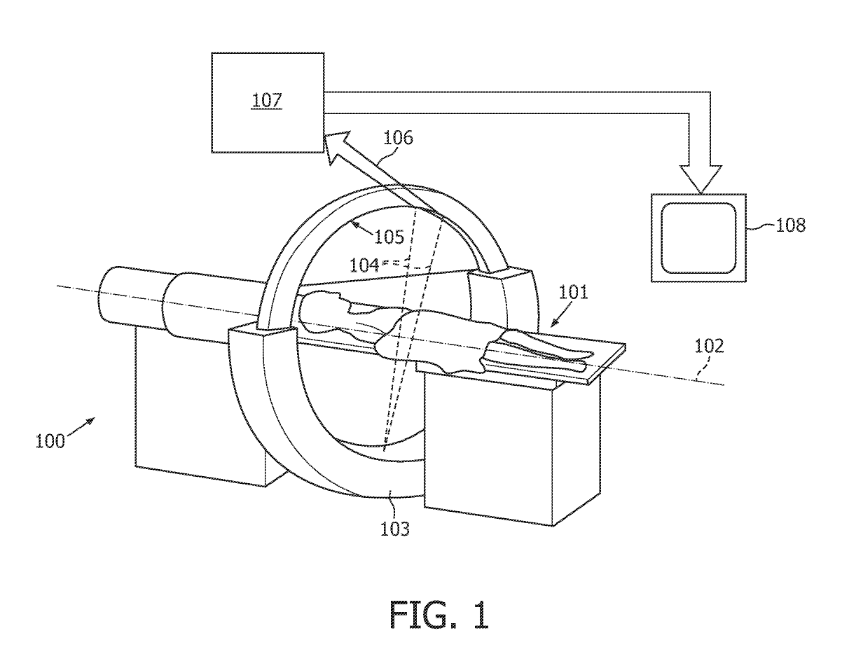

FIG. 1 shows a schematic view of a computer tomography system.

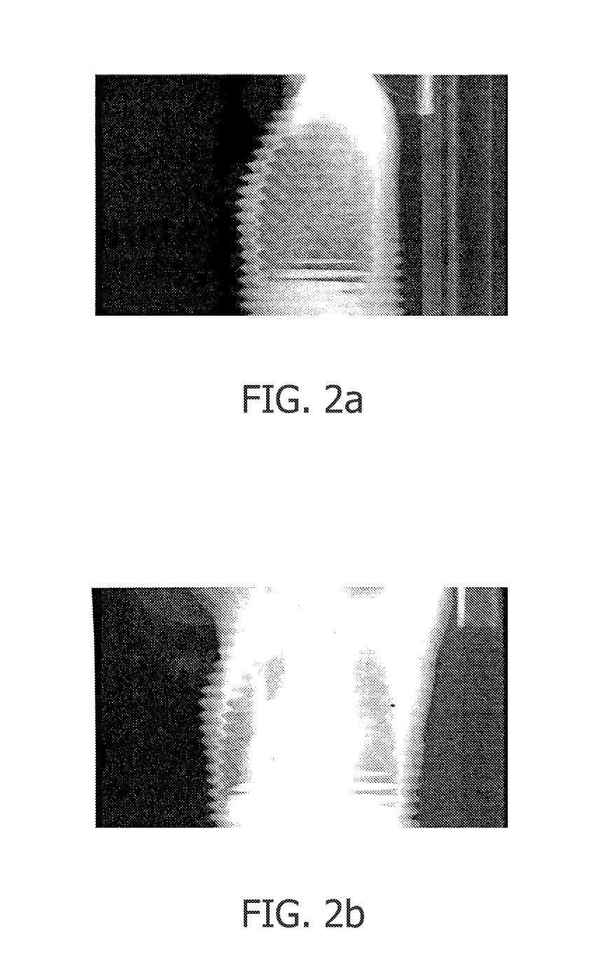

FIG. 2 shows virtual parallel projections of a thorax.

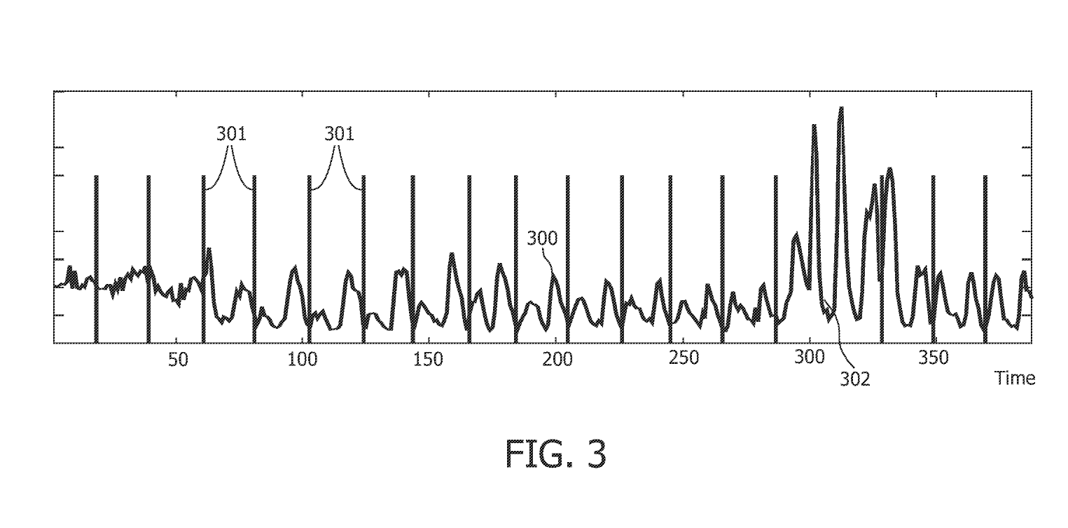

FIG. 3 schematically shows a motion signal.

The illustration in the drawing is schematically. In different drawings, similar or identical elements are provided with the same or similar reference signs.

In the following, referring to FIG. 1, a schematic view of a computer tomography system (CT-system) is shown. The CT-system 100 comprises a support 101 which can support a subject (in FIG. 1 a patient is schematically shown for illustrative reasons). The support 100 is moveable along a longitudinal axis 102 thereof. The CT-system 100 also comprises a housing 103 in which a radiation source, which is not shown in FIG. 1 but which is indicated by radiation beams schematically depicted as dotted lines 104 and which are emitted by the radiation source. The radiation source is rotatable around the support 101 and thus around the subject on the support 101. Furthermore, in the housing 103 a plurality of radiation detectors or radiation sensors are arranged, which are indicated in FIG. 1 by the ring 105. The radiation detectors are arranged in form of a detector array which covers 360°. Furthermore, the CT-system 100 comprises a signal processing unit 107 having an input interface and which receives signal data measured by the detector array 105, which receiving is indicated by the arrow 106.

The signal processing unit 107 further comprises a processor which is adapted to generate a pulmonary gating signal based on the measured data signals. Furthermore the signal processing unit 107 is adapted to generate an image based on said measured data signals by using the pulmonary gating signal, in the following also called gating signal. Thus, the image is generated based on the same data which are used to generate the gating signal. Therefore, an extra sensor whose signals are used to generate a gating signal can be omitted.

The generated image can be displayed on a display which is schematically depicted as 108 in FIG. 1.

FIG. 2 shows virtual parallel projections, which are derived from a low-pitch helical data set, according to a method according to an embodiment of the present invention. The computing of the images was done by performing an intermediate step in which several virtual parallel projections are computed from the helical data. For achieving this virtual parallel projections the helical or cone-beam data are re-binned into wedge geometry. Afterwards virtual parallel projection are extracted by taking a central detector line every 180°, whereby every other view is mirrored and corrected of a detector shift which corresponds to the pitch of the helical data. All these data form the virtual parallel projection of a patient which are shown in two examples in FIG. 2. The image in the upper FIG. 2a shows a side view of a thorax of a patient, i.e. a projection of 90°, while the image in the lower FIG. 2b shows the thorax at a projection angle of 45°. The scan of the thorax was done during approximately 100 seconds. The time scale is represented by the vertical axis (height) in FIG. 2. The projection data for both images were acquired using a pitch of 0.08 and a collimation of 16×1.5 mm. By identification of the maxima of the chest wall the time of the maximum inhalation can be derived. Correspondingly, minima yield the time for maximum exhalation.

FIG. 3 schematically shows a motion signal and illustrate an identifying of maxima and minima of the chest wall. The identifying of the maxima and minima is a two-dimensional processing problem. However, the problem can be simplified to a one-dimensional problem. The x-axis represents the time axis in arbitrary units. The curve 300 represents the motion signal from projections and was obtained by calculating the root mean square difference between succeeding line in the image shown in FIG. 2a. The arrows 301 indicate the detected breathing triggers by a trigger system, e.g. a Varian system. A small difference is obtained at the points of maximum in- and exhalation, but generally the position of every second minimum correlates well with the trigger pulses of the dedicated pulmonary trigger device. However, at the point marked which 302 one breathing cycle was missed by the dedicated breathing sensor. At this time the depth of breathing was strongly changed as can be seen when viewing at the motion signal from the projections.

By looking at the temporal behaviour of the curve 300 it can be concluded that changes of the signal are nicely recovered by our sampling. When using a rotation time of 0.5 seconds, the motion signal has a temporal resolution of 0.25 seconds, meaning that one sample of the curve is taken every 250 ms. Under such circumstances no undersampling takes place. Thus, it is possible to interpolate samples between the original samples in order to estimate the true minima more accurately.

It should be noted that the term “comprising” does not exclude other elements or steps and the “a” or “an” does not exclude a plurality. Also elements described in association with different embodiments may be combined. It should also be noted that reference signs in the claims shall not be construed as limiting the scope of the claims.

1. A tomographic imaging device comprising:
a radiation source;
a radiation detector; and
a processor that generates a pulmonary grating signal based on measured data signals;
wherein the radiation source rotates at a speed which covers a periodic motion of an object under examination.

2. The tomographic imaging device according to claim 1, wherein the pulmonary gating signal relates to minima of a periodic motion of the object; and

3. (canceled)

4. (canceled)

5. (canceled)

6. (canceled)

7. (canceled)

8. The tomographic imaging system according to claim 1, wherein the speed is higher than 1 rotation per second.

9. The tomographic imaging system according to claim 1, wherein the radiation detector measures the data signals every 180° of the rotation of the radiation source.

10. The tomographic imaging system according to claim 1,
wherein the radiation source emits a cone beam.

11. A method for producing an image based on data signals of a computer tomography system the system comprises a tomography device having a radiation source and a radiation detector, and a signal processing unit, the method comprising:
measuring data signals by using the radiation detector, which data signals are based on radiation emitted by the radiation source after having passed an object under examination;
generating a pulmonary gating signal based on said measured data signals; and
generating an image based on said measured data signals by using the pulmonary gating signal;
wherein the pulmonary gating signal is adapted to represent a motion state of the object under examination, wherein the motion state is a minimum of a periodic motion of the object under examination,
wherein the radiation source is rotatable and is adapted to rotate at a speed which is adapted to cover a periodic motion of the object under examination.

12. The method according claim 11,
wherein the measured data signals are cone beam data, and
wherein the method further comprising:
re-binning the cone-beam data into wedge geometry before generating the image based on the measured data signals.

13. The method according to claim 11, further comprising forming a virtual parallel projection of the object under examination.

14. The method according to claim 13,
wherein the pulmonary gating signal is generated based on a plurality of virtual parallel projections.

15. (canceled)

16. (canceled)

17. A computer readable medium in which a program for producing an image based on data signals of a muter tomography system having a radiation source and a radiation detector is stored, which program, when executed by a processor, is adapted to control a method according to claim 11.

18. A program element for producing an image based on data signals of a computer tomography system having a radiation source and a radiation detector, which program, when executed by a processor, is adapted to control a method according to claim 11.
